(19) United States
(12) Patent Application Publication
(10) Pub. No.: US 2012/0123507 A1
(43) Pub. Date: May 17, 2012

(54) Phototherapeutic Apparatus and Method

(75) Inventor: Colin Whitehurst, Cheshire (GB)

(73) Assignee: Photo Therapeutics Limited, Altrincham (GB)

(21) Appl. No.: 13/139,837

(22) PCT Filed: Dec. 15, 2009
(86) PCT No.: PCT/GB2009/002886

§ 371 Date: Feb. 6, 2012

(30) Foreign Application Priority Data
Dec. 15, 2008 (GB) 0822835.5

(51) Int. Cl. A61N 5/06

(52) U.S. Cl. 607/88

(57) ABSTRACT

A photo therapeutic device for treating a patient, comprising: a plurality of discharge lamps arranged to emit light with a wavelength of primarily between 590 and 690 nm and a plurality of diode lamp arrays arranged to emit light with a wavelength length of primarily between 780 and 920 nm, wherein the discharge and the diode lamps are arranged to irradiate at least a substantial part of the length of a patient's body.

### FIELD OF THE INVENTION

The present invention relates to methods of, and apparatus for, phototherapy, in particularly for a device and method for treating a large area of a patient with phototherapy, such as for example photodynamic therapy (PDT), skin rejuvenation, enhancing aesthetic treatments and/or wound healing.

### BACKGROUND OF THE INVENTION

Light therapy has been described to be useful for a variety of purposes, for example photodynamic treatment (PDT) and cosmetic treatment of aged skin, or treatment of wound or sores.

For example, patent application WO 06/013390 relates to methods of skin rejuvenation, whereby the skin is subjected to multiple courses of phototherapeutic treatment using non-laser near-infrared light over a predetermined period of between several days and up to 10 weeks. In an alternative phototherapeutic method the patient is treated in two courses of phototherapy using red and/or infrared light. The method may enhance an aesthetic treatment which relies on photothermolysis or mechanical damage. In another method, a course of phototherapy comprising discrete sessions of phototherapy, using red and infrared light separately, is used to improve wound healing.

U.S. Pat. No. 5,800,479 describes a method of treatment of wounds or sores using pulsed infrared and visible light emitted by an LED array. In one example, the pulsed infrared and visible light alternate over a period of between one and three minutes. The preferred wavelength of the visible light is 660 nm.

In the following, the terms “phototherapy” and “phototherapeutic” are used to include any type of light treatment, including cosmetic and aesthetic treatments.

Today, there are many different types of phototherapy devices available. The emitted light of these devices is chosen to match the target chromophores (eg photoacceptors) and the resulting efficacy depends on the precise choice of wavelength, dose rate and light dose.

However, the devices are designed for treatment of small- to medium-sized areas such as the face, scalp or chest. The reason for this is twofold: Firstly, the customers usually consider these local areas to have the highest priority for phototherapy such as rejuvenation as they are the most visible and, therefore, from a commercial point of view, phototherapy devices for treatment of small- to medium-sized areas are expected to have the highest demand and thus also the highest commercial revenue.

The second reason is that devices designed for treatment of large areas are technically challenging and a significant increase in costs is therefore expected if significantly increasing the therapeutic area.

For phototherapy, and for photorejuvenation in particular, efficacious light sources are usually used with a light output limited to a broadband, or even a narrowband emission around key wavelengths such as 630 nm and 830 nm. These emissions can be produced using, for example, filtered white light sources (such as for example incandescent bulbs, Xenon arc lamps, Intense Pulsed Light (IPL) lights, fluorescent tubes), low-intensity narrow or broadband discharge tubes, or light emitting diodes (LEDs). The most effective and efficient of these light sources are LEDs as they can be carefully selected to produce only those wavelengths and intensities which have been proven to be beneficial for phototherapy, such as photorejuvenation, wound healing, pain control, etc. However, to scale the therapy up to significantly larger areas, such as whole body or a significant area thereof, poses problems for all the above-mentioned sources.

Scaling up the commonly used light sources such as filtered white light for large area treatment would result in the necessity for a very large power supply and consequently the generation of an excessive amount of heat in close proximity to the client during phototherapy. Similarly, expansion of the target area for treatment to cover a large area when using discharge tubes would be very costly and would, only produce a very low intensity incapable of delivering a sufficient light dose within an acceptable time interval and within the critical bandwidth. Even the wavelength-selected LEDs, which are ideal for illuminating small to medium areas, would be very expensive if the same optimal parameters of intensity and wavelength were to be scaled up to cover a larger area. In addition, manufacturing such a device suitable for phototherapy of a large area or the whole body of a patient would be very costly due to the need of individually placing of up to tens of thousands of LEDs and their respective drive circuits.

It is therefore an aim of the invention to alleviate at least some of the aforementioned problems.

It is another aim to provide a technology better suited to the production of large quantities of light over a large area, such as the whole body, which is still capable of providing the required wavelength, dose rate and light dose. It is another aim to provide an efficient light source with a reduced heat generation suitable for large area phototherapy. It is another aim to provide a cost effective phototherapeutic light source suitable for an increased illumination area.

### STATEMENT OF THE INVENTION

According to one aspect of the present invention, there is provided a phototherapeutic device for treating a patient, comprising: a plurality of discharge lamps arranged to emit light with a wavelength of primarily between 590 and 690 nm and a plurality of diode lamp arrays arranged to emit light with a wavelength of primarily between 780 and 920 nm, wherein the discharge and the diode lamps are arranged to irradiate at least a substantial part of the length of a patient's body.

According to another aspect of the present invention, there is provided a phototherapeutic device for treating a patient, comprising: a plurality of diode lamp arrays arranged to emit light with a wavelength of primarily between 780 and 920 nm, wherein the diode lamp arrays are arranged to irradiate at least most of the front and the back over at least a substantial part of the length of a patient's body.

According to another aspect of the present invention, there is provided a photo therapeutic device for treating a patient, comprising: at least one discharge lamp arranged to emit light with a wavelength of primarily in a first region, and at least one diode lamp array arranged to emit light with a wavelength of primarily in a second region.

According to another aspect of the present invention, there is provided a photo therapeutic device for treating a patient, comprising: a plurality of discharge lamps arranged to emit light with a wavelength of primarily between 590 and 690 nm and wherein the discharge lamps are arranged to irradiate at least most of the front and the back side of a patient over at least a substantial part of the length of a patient's body.

According to another aspect of the present invention, there is provided a photo therapeutic device for treating a patient, comprising: a plurality of first lamps arranged to emit light with a wavelength of primarily between 590 and 690 nm and a plurality of second lamps arranged to emit light with a wavelength of primarily between 780 and 920 nm, wherein the first and second lamps are arranged to irradiate at least a substantial part of the length of a patient.

Specific embodiments of the present invention will now be described with reference to the accompanying drawings, in which:

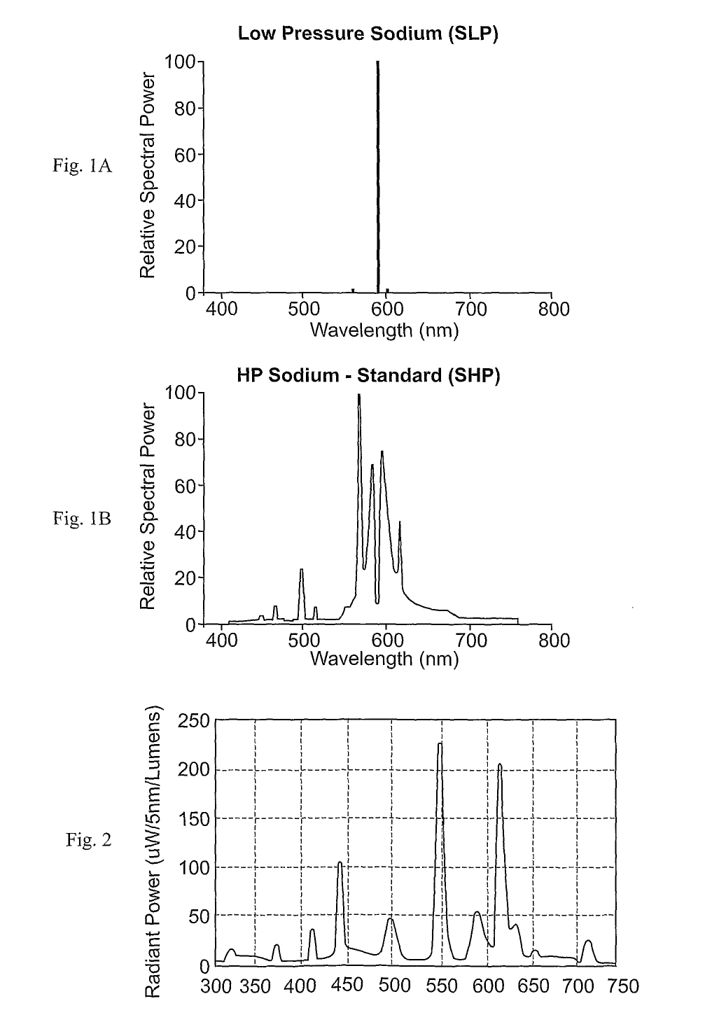

FIGS. 1A and 1B show the emission spectrum of LPS and HPS, respectively;

FIG. 2 shows the emission spectrum of a metal-complex lamp doped with Zinc (Zn);

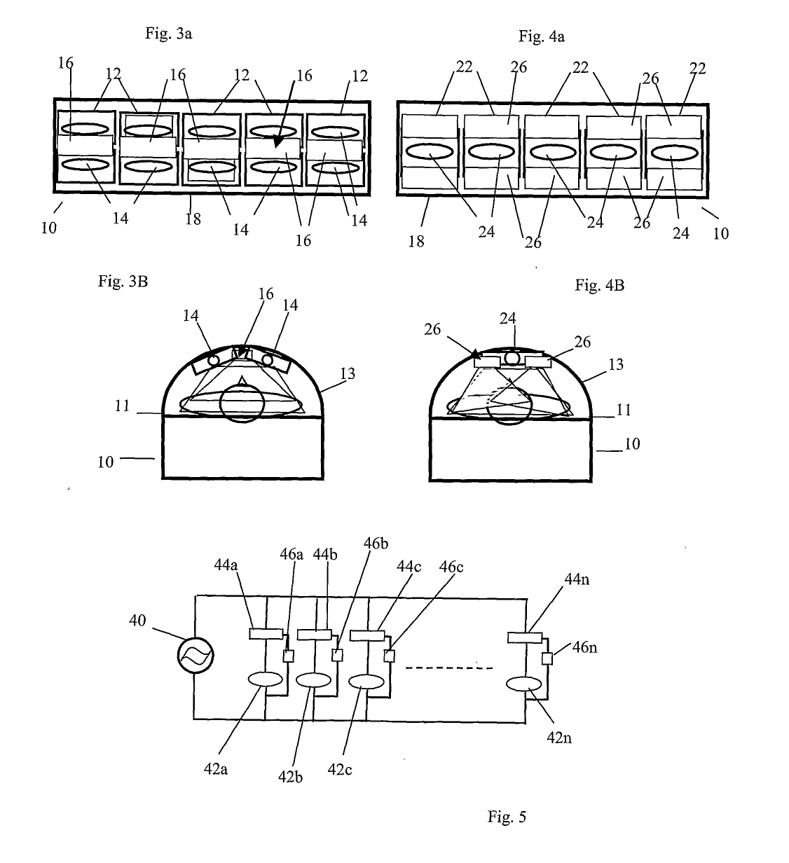

FIGS. 3A and 3B are respectively side and frontal schematic views of a treatment bed of a patient's body according to a first embodiment of the present invention;

FIGS. 4A and 4B are respectively side and frontal schematic views of a treatment bed of a patient's body according to a second embodiment of the present invention;

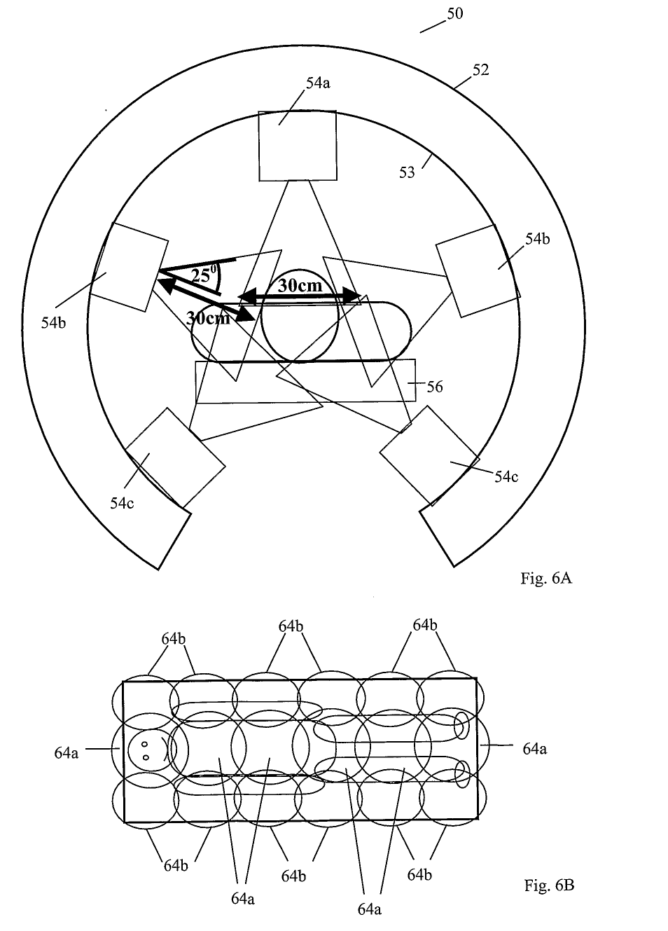

FIG. 5 is a selective circuit diagram for a HPS/LPS lamp array according to an embodiment of the present invention;

FIGS. 6A and 6B are respectively schematic front and top views of a treatment bed for a patient's body according to a seventh embodiment of the present invention.

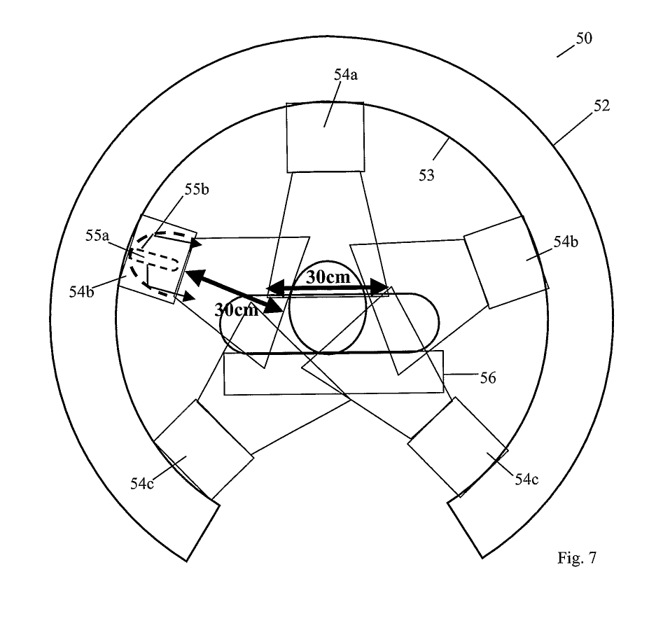

FIG. 7 is a schematic front view of a treatment bed for a patient's body according to an eighth embodiment of the present invention.

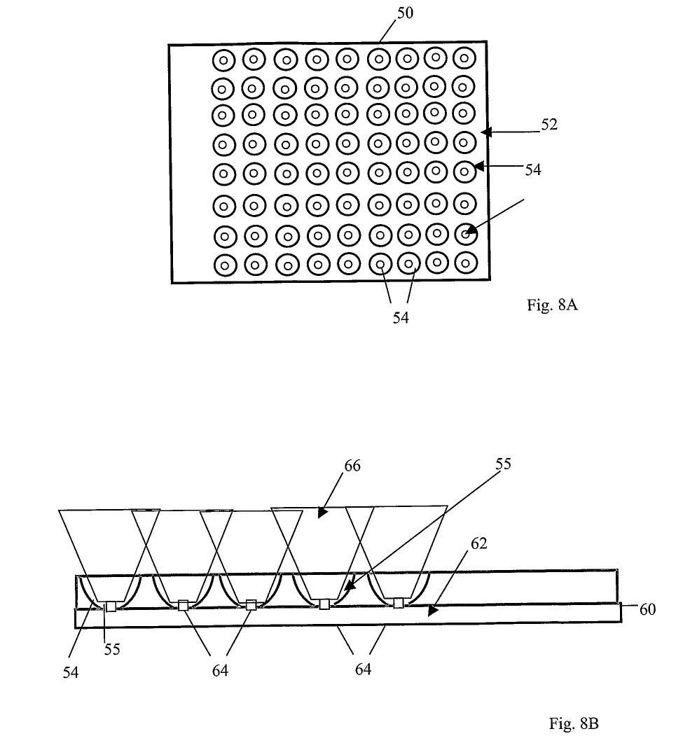

FIG. 8A is a schematic diagram of a metal reflector array containing near-infrared LEDs according to a further embodiment of the present invention; and

FIG. 8B is a schematic cross-section of the metal reflector array of FIG. 6A.

### EMBODIMENTS OF THE PRESENT INVENTION

In the following a phototreatment device and method will be described which is suitable for phototherapy of a large area or the whole body of the patient.

Visible red light and invisible, near-infrared light has been described as being particularly efficient for the treatment of the above described conditions.

The key wavelength for phototreatment corresponding to visible red light is in the interval between 590 nm and 690 nm, especially between 620-640 nm, and most efficiently at around 630 nm.

The second key wavelength, corresponding to invisible, near-infrared light, is in the range from 780 to 920 nm, and between 820 and 860 nm in particular, and most efficiently at around 830 nm and/or 850 nm.

As described above, one of the key wavelengths for phototreatment or therapy such as photorejuvenation is around 630 nm (corresponding to visible red light), and there are very few powerful sources in this spectral region that would be cost effective and/or suitable for a phototreatment device for a large area of the patient's body, such as for example a rejuvenation bed. The second key wavelength is around 830 to 850 nm, i.e. invisible, near-infrared light. Usually semiconductor light (diodes) are used to produce the light of this wavelength.

### High Pressure Sodium (HPS) and Low-Pressure Sodium (LPS) Lamps/Luminaires

Alternative light sources for emitting light at a wavelength in the region of 630nm are high pressure sodium (HPS) discharge lamps or low pressure sodium (LPS) discharge lamps. As will be described in more detail below, sodium discharge lights are suitable for phototreatment of a wavelength in the region of 630 nm, as they are both cost effective and suitable for treatment of a large area of a patient's body. The sodium discharge lamps can be used either isolated or in a self-contained reflector housing (luminaire).

Sodium discharge lamps are some of the most efficient light sources known with HPS efficiencies of 30-50% and LPS efficiencies of up to 60%, and most of their spectral emission is concentrated between 570 nm and 640 nm. In addition, these lamps are readily available at low cost. HPS lamps are commonly used for security lighting of large outdoor areas, whereas LPS are used as street lighting.

HPS and LPS lamps are suitable for photodynamic therapy (PDT), where emission around the 570 nm-670 nm region is critical for the stimulation of either exogenous or endogenous chromophores, but also for the treatment of skin conditions such as cancerous or precancerous lesions, photorejuvenation (ie treating photodamaged skin or reversing the effects/signs of ageing), acne, wrinkle reduction or wound healing.

FIGS. 1A and 1B illustrate the emission spectrum of LPS and HPS lamps, respectively.

As can be seen from FIG. 1B, although there are a few minor emission bands between 400 nm and 550 nm, the vast majority of the HPS emission spectrum is centred around 590 nm with an asymmetrical emission bandwidth of about 70 nm. It is noted that the emission bands between 400 nm and 500 nm can be filtered out, as will be discussed in more detail below. In contrast, the whole of the LPS emission spectrum is centred at 590 nm, as can be seen in FIG. 1A.

### High Pressure Sodium Lamps

The HPS bandwidth is approximately twice the FWHM (full width half max) bandwidth of LEDs currently used for photorejuvenation or PDT. Although the bandwidth of a HPS lamp is broader than that of LEDs and will therefore have a reduced efficacy, the wavelengths included at the extremities of the emission band are only slightly disadvantageous compared to the bandwidth of the LEDs commonly used for phototherapy. This is because the chromophores involved in both PDT (for example in therapy using Aminolaevulinic Acid (ALA)) and also for photorejuvenation and wound-healing (for example photoacceptors, cytochrome c) can still be stimulated at these peripheral wavelengths, albeit to a lesser extent. Thus, the emission spectrum of a HPS lamp can be used for phototherapy without the need of alterations.

Alternatively, the emission band can be narrowed down and thus made more selective. In this way the treatment efficacy is increased. Reduction of the width of the wavelength band can be achieved by using a filter. For example, either a sheet of low-cost, polycarbonate, long-pass filter or a higher specification, dielectric, long-pass filter can be used and placed in front of the HPS lamp to block the shorter wavelengths. For example, the filter may be selected so as to block the wavelengths up to 560 nm. Alternatively, the emission spectrum can be narrowed down even further, by blocking the wavelengths up to 590 nm. However, the use of a filter results not only in a narrower, more selective spectral emission, but also in a reduced overall output.

Therefore, it is possible to use these shorter wavelengths in phototherapy such as photorejuvenation, as described above, and a possible treatment method includes these shorter passbands, i.e. the emission spectrum of the HPS lamp of a wavelength below 560 or 590 nm. In this way the use of light flux emitted by the lamp, is maximised, and the number of lamps required minimised.

### Low Pressure Sodium Lamps

Also LPS lamps can be used as phototherapeutic light sources. LPS lamps are extremely efficient and no filtering is required as the LPS emission spectrum is concentrated at 590 nm (see FIG. 1A). However, the location of the narrow emission peak is close to but does not match the peak absorption band of key chromophores.

Phototreatment with Sodium Discharge Lamps

For a light with a suitable spectral output, a light intensity of at least 10 mW/cm² is required for effective phototreatment (such as photorejuvenation) within an acceptable illumination time, and preferably above 50 mW/cm².

Typically, HPS has a lamp conversion efficiency of at least 33%. Thus, a 150 W lamp emits 50 W light, a 300 W emits 100 W light and a 400 W lamp emits 133 W light. Therefore, assuming an intensity of 50 mW/cm² each lamp/luminaire could illuminate an area of approx 25 cm×40 cm (for 150 W) or 50 cm×40 cm (for 300 W) and 60 cm×45 cm (for 400 W), assuming all the output from the lamp was utilised using reflectors. Each of the lamps could have their own individual back-reflector or they could use a common reflector of the type normally built into sunbeds. The use of sodium discharge lamps in phototreatment devices suitable for a large area or whole body treatment will be described in more detail below.

### High-Pressure, Metal-Transition-Complex, Halide Discharge Lamps

An alternative light source to sodium discharge lamps which is also suitable for use in phototherapy and emits red light of a wavelength around 630 nm is a high pressure halide lamp.

The emission in the red region of a high-pressure metal-halide lamp, is greatly increased by doping with zinc or scandium iodide. The emission spectrum of a metal complex lamp doped with zinc (Zn) is shown in FIG. 2.

If Zn/Sc iodide is added to a high-pressure metal-halide discharge lamp, there can be seen an enhancement of lines of the zinc (472, 481 and 636 nm) and a molecule continuum (B-X band system of the zinc iodide) with a maximum “satellite” at 602 nm. The emission can be further enhanced by the addition of thallium iodide. Doping with Th transfers most of 450 nm, 500 nm and 550 nm pressure-broadened lines to the 600-640 nm band.

The short-red can be enhanced further by adding a red emitter (eg calcium iodide). The calcium iodide emits primarily along a two-band systems (A-X: around 640 nm, B-X: around 630 nm), resulting in an increase in the output in the red region of the spectrum whereas the emission spectrum output in the short blue continuum is decreased. Alternatively, for the use in photodynamic therapy the emission of light of short blue wavelength is removed. Thus the addition of the red-emitter therefore decreases the colour temperature Tc.

### Treatment Bed Configuration

### First Embodiment

FIGS. 3a and 3b are schematic front and side views of an example of a full-body, phototherapy bed equipped with high-pressure discharge lamps.

The treatment bed 10 shown in FIG. 3 is constructed similar to a sun bed. It includes a bed 11 supporting the patient in a recumbent position and a housing 13. The housing 13 supports phototherapy light sources which are mounted on an upper part of the housing, such that the whole of one side of the body of the patient can be treated.

The light sources are arranged in five arrays 12. These arrays 12 are arranged in line to cover the length of bed 11 for treating the body of the patient. All five arrays together irradiate the whole upper side of the body of the patient. Each of the arrays 12 includes three lamps or lamp arrays. A semiconductor light source 16 is arranged in the centre of the array 12, such that it extends over the whole length of the array 12 in a direction along the length of the patient, although the length can vary. In this way it is ensured that the lamp irradiates the whole or the majority part of the patient covered by array 12. Two sodium discharge lamps are arranged on both sides of the semiconductor light source 16, such that the two discharge lamps together irradiate the whole part of the patient covered by array 12.

As the discharge lamps have an isotropic output, back reflectors are used in order to increase light flux in the direction of the patient. In the present embodiment back reflectors such as the reflectors commonly used in sunbeds or other phototherapy devices are used. Alternatively, individual luminaires which are custom built for each lamp and each lamp type may be used.

The phototherapy device also includes filters to remove wavelengths shorter than a predetermined wavelength, for example, 560 nm. In the present embodiment either long-pass, low-cost, polycarbonate filters or dielectric filters are used. In this way the efficacy of the device is further increased. Each array includes a long-pass polycarbonate or dielectric filter sheet 18.

The filter may be removable such that the device can be used either with or without the filter. In addition, alternative and or interchangeable filter sheets may be provided, arranged to remove wavelengths shorter than 590 nm. In this way the phototherapy device can be adopted to the requirements of the method required for a certain therapy.

In one embodiment 150 W sodium discharge lamps are used. Assuming a lamp conversion efficiency of 33%, (ie a 150 W lamps emits 50 W light), then each lamp/luminaire is selected to illuminate an area of approximately 25 cm×40 cm. As each array 12 includes two of the sodium discharge lamps each array is capable to illuminate an area of approximately 50 cm×40 cm. Thus, a total of five arrays 12 is sufficient to irradiate the whole of the patient.

In an alternative embodiment 150 W lamps are used. In this case each luminaire is selected to illuminate an area of approximately 25 cm×40 cm, and a total of ten arrays 12 is used for a whole body phototreatment bed.

### Second Embodiment

FIG. 4 is a schematic diagram of an alternative whole body phototreatment bed. The embodiment is very similar to the one shown in FIG. 3, and the same reference numerals are used for the same parts of those of FIG. 3. However, the phototherapy apparatus 10 includes arrays 22a for treatment of the patient.

In each of the array 22 of the treatment apparatus of FIGS. 4A and B a discharge lamp 24 is arranged in the centre of each array 22. Two semiconductor arrays 26 are arranged on both sides of the sodium discharge lamps 24. Again, the lamps are arranged so that both the one discharge lamp and also the two semiconductor lamps together irradiate the whole part of the patient covered by array 22.

In the second embodiment 300 W sodium discharge lamps are used.

Assuming a lamp conversion efficiency of 33%, (i.e. a 300 W lamps emits 100 W light), then each lamp/luminaire is selected to illuminate an area of approximately 50 cm×40 cm. Thus, a total of five arrays 22 may be sufficient to irradiate the whole of the patient.

In an alternative embodiment 150 W lamps are used. In this case each luminaire is selected to illuminate an area of approximately 25 cm×40 cm, and a total of ten arrays 22 is used for a whole body photo treatment bed.

FIG. 5 illustrates a schematic circuit diagram for discharge lamps for the photo treatment apparatus of the above described embodiments. The circuit includes a mains power supply 40 of 110 or 240V, and discharge lamps 42a, 42b, 42c, . . . 42n. Each of these discharge lamps 42i is connected in parallel to power supply 40. In addition, connected to each discharge lamp is an igniter 44i and a capacitor 46i.

It is understood that for the discharge lamps described in the first and second embodiments either HPS or LPS lamps can be used.

### Third and Fourth Embodiment

Two further embodiments are similar to the first and second embodiments described above. However, instead of sodium discharge lamps metal halides lamps are used as those described above.

### Fifth and Sixth Embodiment

Two further embodiments are again similar to the first and second embodiments described above. However, instead of sodium discharge lamps diode lamps emitting red light are used, for example at a wavelength primarily between 590 and 690 nm. Thus the device includes two different types of diode lamps to irradiate a patient with NIR light and/or with red light.

### Seventh Embodiment

In the following a further embodiment of a treatment bed will be described with reference to FIG. 6A. This device uses either NIR or red emitting diode lamps to irradiate the patient.

The device 50 includes a housing 52 in a shape of a part of a tube or an open tubular framework. Attached to the housing 52 is a bed 56 for the patient. The bed 56 is made of a translucent material, such as acrylic plastic. The housing includes a movable portion (not shown) such as a door, such that the patient can enter and exit the housing 52. Alternatively, the bed 52 is slidable, such that the bed 56 can be moved outside the housing 52. The patient can then comfortably recline onto the bed when the bed is moved outside the housing, and the patient lying on the bed 56 can then be slided into the housing 52. Alternatively the upper section of the open tubular framework 52 can be raised on a hinge as in a standard sunbed and the patient simply reclines onto the bed and closes the hinged lid behind them.

The housing supports a plurality of diode lamps 54 for irradiating the patient. A subset of five lamps (or lamp arrays) 54 are mounted in a circumferential direction to the (length of the) patient on the inner side 53 of the housing 52. Each of the five lamps of the subset is arranged in a plane perpendicular to the length of the patient. One lamp 54a of the subset is mounted on the lower side at the top of the housing 52, such that it irradiates a patient laying on the treatment bed 56 from above. Two further lamps 54b are mounted at an angle of about 70° to both sides of the lamp 54a. Two further lamps 54c are mounted at an angle of about 135° to both sides of the lamp 54a.

Altogether six of these subsets of lamps are arranged along the length of the patient to irradiate substantially the whole length of the patient. The six subsets of lamps are spaced apart by about 30 cm, so that the six subsets of lamps irradiate a length of about 180 cm.

Each of the lamps (or lamp arrays) emits light radially in a cone with an opening angle of about 25 to 35 degrees. The lamps are arranged to irradiate the patient at a distance of about 30 cm. At this distance, the beam spot of the diode lamp light has a diameter of about 30 cm.

Such a lamp can be achieved for example by using a lamp array of diode lamps whereby the diodes are placed centrally at the bottom of circular wells formed in the substrate for the purposes of collecting the light emitted by the diode lamps and concentrating it in a direction towards the patient. In this case the divergence of the lamps can be achieved by selecting the shape of wells in the diode substrate. Alternatively, a reflector dish can be used to achieve the desired shape and divergence of the lamps or lamp arrays, as is well-known in the art, or a metal reflector array may be used as is described in more detail below.

By using a treatment bed as described in this embodiment both the upper and lower half side (i.e. the front and the back of the patient) can be treated at the same time.

FIG. 6B is a schematic top view showing the projected beams of the lamps 54a and 54b. This figure schematically shows how the lamps 54 irradiate the front side of the patient.

It is understood that alternative designs are possible. For example, the angle between the lamps 54a, 54b and 54c can be varied. According to an alternative to the seventh embodiment the five lamps 54 of the subset are mounted with approximately equal spacing at an angle of about 70° to each other. In an alternative arrangement, the angular spacing is variable, i.e. not constant all the way around the patient. In a further alternative each subset includes six lamps. Also the number of subsets used for the whole treatment bed can be varied. In a further alternative seven subsets of lamps are used to provide a treatment bed of greater length.

Also, if lamps with a different intensity or if lamps with a different opening angle are used, the total number of lamps (i.e. the number of subset and/or the number of lamps per subset) needs to be varied. For example, in a yet further alternative to the seventh embodiment each lamp 54 is of lower intensity, thus the number of subsets and the number of lamps per subset is higher than described above and the spacing between the circumferentially arranged lamps is varied accordingly.

### Eighth Embodiment

In the following a further embodiment of a treatment bed will be described with reference to FIG. 7. This embodiment is very similar to the seventh embodiment discussed above with reference to FIGS. 6A and 6B, and the same reference numerals are used for the same parts of those of FIG. 6A. However, sodium lamps as those discussed above, rather than diode lamps, are used to irradiate the patient with essentially red light in the treatment bed of the eighth embodiment. Each of the lamps 54 include the discharge lamp 55a itself and a reflector 55b. The reflector shapes the output light beam to the desired shape.

### Ninth Embodiment

A further embodiment is essentially a combination of the seventh and eighth embodiments described above. Diode lamps are used for producing NIR light, and discharge lamps are used to produce red light as is described in more detail above.

In this way the treatment bed includes a arrangement of lamps irradiating the patient's body with light in the NIR and red region, similar to the first and second embodiments described above. However, with the arrangement of this embodiment, both the front and the back of the patient's body can be irradiated at the same time.

### Tenth Embodiment

A further embodiment is essentially another combination of the seventh and eighth embodiments described above. Here, diode lamps are used for producing NIR light, and a different diode lamp also for producing red light.

In this way the treatment bed includes a arrangement of lamps irradiating the patient's body with light in the NIR and red region, similar to the first and second embodiments described above. However, with the arrangement of this embodiment, both the front and the back of the patient's body can be irradiated at the same time.

### Metal Reflector Array

It has been described above that diode (LED) light sources are very efficient for photo treatment, but that the use of LED lamps for large area treatment is technically difficult. In the following a Multiple Near Infra Red (NIR) die Metal Reflector Array (MRA) is described which facilitates the use of LED light sources in large area photo treatment.

A reflector array i.e. an array of wells as mentioned above, is used to concentrate the light emitted by the diodes on an area to be treated, and can thus facilitate the use and enhance the efficiency of LEDs in large area photo therapeutic treatment.

FIG. 8A illustrates an example of a metal reflector array according to one embodiment of the present invention. The metal reflector 150 includes a 2-dimensional sheet of aluminium 152. An array of concave reflector cavities or wells 154 is machined into a sheet of aluminium. Each of the cavities 154 has a diode placed centrally at the bottom and has a diameter depending on the divergence of light required. For example a diameter of 3 mm may be selected to emit light with a full width half maximum (FWHM) divergence of 70°. According to alternative embodiments, each cavity is arranged to emit light with a FWHM divergence between 20′ and 120°.

The aluminium sheet 152 has a width of 48 mm and a length of 38 mm. The metal reflector 150 includes 81 cavities 152 blanked into the aluminium sheet 152. Each cavity has a hole 155 in its centre.

The sheet 152 is then overlaid onto a monolithic LED array 60 as illustrated in FIG. 8B. The LED array 60 includes a substrate board 62 containing an array of LEDs 64 emitting near-infrared (NIR) light 66. The pattern of the reflector array 150 matches the LEDs 64 on the board such that each cavity 152 accommodates an LED lamp 64 in its focus.

The metal reflector array 150 with the NIR LEDs 64 produces a monolithic array capable of delivering high power that can be efficiently cooled, thus maintaining high efficiency. In the above described embodiment the array delivers 36 Watts at an efficiency of 25%-30%.

The above described embodiment of the reflector array 150 provide a high efficiency and output power, and is thus particularly suitable for treating a large area or the whole body for PDT or photo therapy. An NM emitter array as described above can be built to order or easily adapted according to the needs for a particular phototherapy apparatus. The reflector array is easy and cost efficient to manufacture. Also, the manufacturing costs for NIR emitters as described above are relatively low due to the low numbers of arrays required. In addition, the thermal stress on the customer can be reduced by the use of the above described reflector arrays.

The above described embodiment of the reflector array 150 is but one example. The diameter of each cavity or the number of diodes per array can vary depending on the optical geometry and power required.

The treatment requires an NIR intensity of at least 10 mW/cm² and preferably greater than 30 mW/cm². Therefore, at 30 mW/cm², each aforementioned LED array could illuminate an area of approximately 36,000/30=1200 cm². A total treatment area suitable for whole body treatment is about 60 cm by 178 cm (24″ by 70″). The NIR lamps are to irradiate the same area as the red lamps is equivalent to about 10,700 cm². Therefore, about 10 MRAs would be required.

### Treatment Method and Light Intensities and Doses

The intensities required for phototherapy is for both key wavelengths (i.e. for wavelength of 830/850 nm and 630 nm) in the range of 1 to 1000 mW/cm², and in particular in the range of 1 to 120 mW/cm². The required light dose for both wavelengths is in the range of 1 to 200 J/cm², and in the range of 1 to 120 J/cm² in particular.

Both pulsed or continuous wave light output is efficient for treatment. Typically, individual treatments with the above intensities and light doses take between 10 seconds to 2 hours. More details of treatment methods may for example be found in patent application WO 06/013390.

The photo therapy devices described in the above embodiments are suitable for photorejuvenation treatment of chronologically- or photo-aged skin, both with or without endogenous or exogenously applied photosensitiser (i.e. PDT-based photorejuvenation), for the reduction of wrinkles using 830 nm/850 nm and/or 630 nm light, for reduction or control of pain using 830 nm light, as for example the reduction of pain during PDT treatment of psoriasis or analgesic effects in many clinical applications, for wound healing of damaged tissue or sports-related injuries using 830 nm and/or 630 nm light, for treatment of skin ulcers, for palliation of psoriasis and treatment of all grades of acne. In addition, the device and method is suitable for enhancement of photothermolysis. The device is suitable for treatment of the above described conditions in humans or animals.

It is to be understood that the various aspects of the different embodiments described above may be implemented individually or in any possible combination.

It is to be understood that the embodiments described above are preferred embodiments only. Namely, various features may be omitted, modified or substituted by equivalents without departing from the scope of the present invention, which is defined in the accompanying claims.

1. A photo therapeutic device for treating a patient, comprising: a plurality of discharge lamps arranged to emit light with a wavelength of primarily between 590 and 690 nm and a plurality of diode lamp arrays arranged to emit light with a wavelength of primarily between 780 and 920 nm, wherein the discharge and the diode lamps are arranged to irradiate at least a substantial part of the length of a patient's body.

2. A device as claimed in claim 1, wherein the lamps are arranged within a housing, and the device comprises an aperture allowing at least the substantial part of the patient's body to be inserted into the housing.

3. A device as claimed in claim 1, wherein the lamps are arranged in a plurality of arrays arranged in line with the length of the patient's body.

4. A device as claimed in claim 1, wherein each of the arrays comprises at least one of the discharge lamps and one of the diode lamp arrays.

5. A device as claimed in claim 1, wherein the discharge lamps are either of low pressure sodium, high pressure sodium or metal halide discharge lamps.

6. A device as claimed in claim 1, wherein the arrays are mounted on the inner surface of a housing.

7. A device as claimed in claim 1, wherein the device comprises one or more filter sheets.

8. A device as claimed in claim 1, wherein at least one of the diode lamp arrays comprises a reflector to concentrate the light emitted by the diode lamps on an area to be treated.

9. A device according to claim 1, wherein the reflector comprises a plurality of cavities machined into a metal sheet or substrate.

10. A device according to claim 1, wherein the reflector comprises an array of diode lamps, wherein each diode lamp is accommodated in one of the cavities of the reflector sheet.

11. A device according to claim 1, wherein each of the LED lamps accommodated in cavities of the reflector emits light with a full width half maximum divergence between 20° and 120°.

12. A device according to claim 1, wherein each of the LED lamps accommodated in a cavities of the reflector emits light with a full width half maximum divergence of about 70°.

13. A device as claimed in claim 3, wherein at least one of the further arrays comprises two arrays of diode lamps and one discharge lamp.

14. A device as claimed in claim 13, wherein the discharge lamp is arranged in the centre of the array, and the two diode lamp arrays are arranged on each side of the discharge lamp.

15. A device as claimed in claim 3, wherein at least one of the further arrays comprises one array of diode lamps and two discharge lamps.

16. A device as claimed in claim 15, wherein the diode lamp array is arranged in the centre of the array, and the two discharge lamps are arranged on each side of the diode lamp array.

17. A device as claimed in claim 3, wherein the discharge lamp(s) of the arrays are arranged to irradiate an area corresponding to a part of the length of the patient.

18. A device as claimed in claim 3, wherein the diode lamp arrays are arranged to irradiate the whole of the area corresponding to a part of the length of the patient.

19. A device according to claim 1, wherein a subset of the discharge and/or diode lamps are arranged in a substantially circumferential direction to the patient.

20. A device according to claim 19, wherein the discharge and/or diode lamps comprise a plurality of subsets, and each subset of lamps are arranged in a direction substantial circumferential to the patient.

21. A device according to claim 20, wherein each subset comprises five lamps.

22. A device according to claim 20, wherein the discharge and/or diode lamps in each subset are arranged at an angle of about 60° to 100° to each other.

23. A device according to claim 20, wherein one of the lamps in each subset is arranged vertically above the patient.

24. A device according to claim 20, wherein a subset of the lamps are arranged in a plane perpendicular to the'length of the patient.

25. A photo therapeutic device for treating a patient, comprising: a plurality of diode lamp arrays arranged to emit light with a wavelength of primarily between 780 and 920 nm, wherein the diode lamp arrays are arranged to irradiate at least most of the front and the back over at least a substantial part of the length of a patient's body.

26. A photo therapeutic device for treating a patient. comprising: at least one discharge lamp arranged to emit light with a wavelength of primarily in a first region, and at least one diode lamp array arranged to emit light with a wavelength of primarily in a second region.

27. A device according to claim 26, wherein the at least one discharge lamp is arranged to emit light with a wavelength of primarily between 590 and 690 nm and/or the at least one diode lamp array arranged to emit light with a wavelength of primarily between 780 and 920 nm.

28-37. (canceled)

38. Use of a device according to claim 1 in the treatment of one or more of: photorejuvenation of chronologically- or photo-aged skin with or without endogenous or exogenously applied photosensitiser, reduction of wrinkles, reduction/control of pain, wound healing of damaged tissue or sports-related injuries, treatment of skin ulcers, palliation of psoriasis, treatment of all grades of acne, enhancement of photothermolysis.

39. Use of a device according to claim 1 in the treatment of humans or animals.

40. Use according to claim 38, wherein the affected area of the patient is treated with a photosensitizer.

41. A method of cosmetic treatment of a human or animal body, comprising:
applying a photosensitizer to the area to be treated, and illuminating the area with light from a device according to claim 1.

42. A method of treatment of a human or animal body, comprising: illuminating the area with light from a light source according to claim 1.

43. (canceled)
